(19) United States
(12) Patent Application Publication
(10) Pub. No.: US 2021/0113914 A1
(43) Pub. Date: Apr. 22, 2021

(54) A GAIT CONTROLLED MOBILITY DEVICE

(71) Applicant: Nimbus Robotics, Inc., Pittsburgh, PA (US)

(72) Inventor: Xunjie Zhang, Pittsburgh, PA (US)

(73) Assignee: Nimbus Robotics, Inc., Pittsburgh, PA (US)

(21) Appl. No.: 17/051,573

(22) PCT Filed: Apr. 29, 2019
(86) PCT No.: PCT/US2019/029742

§ 371 Date: Oct. 29, 2020

Related U.S. Application Data
(60) Provisional application No. 62/664,203, filed on Apr. 29, 2018.

(51) Int. Cl. A63C 17/12; A63C 17/14; A63C 17/04; G05D 1/02

(52) U.S. Cl. CPC A63C 17/12; A63C 17/1409; A63C 17/04; G05D 1/0223; G05D 1/0231; G05D 1/0255; G05D 1/0214

(57) ABSTRACT

A mobility device comprising a motorized shoe to be worn by a user to increase the speed of walking. The motorized shoe has a plurality of wheels, with at least one wheel driven by an electric motor through a geartrain. On onboard controller gathers data from at least one of an inertial measurement unit, an ultrasonic sensor, and a vision system to generate a command speed to the electric motor. A user wearing a pair of the mobility devices, one on each foot, is able to walk with a normal gait, but at an increased speed.

### CROSS-REFERENCE TO RELATED APPLICATIONS

This application claims the benefit under 35 U.S.C. § 119 of Provisional Application Ser. No. 62/664,203, filed Apr. 29, 2018, which is incorporated herein by reference.

### STATEMENT REGARDING FEDERALLY SPONSORED RESEARCH

Not applicable.

### BACKGROUND OF THE INVENTION

Embodiments of the present invention relate to the field of mobility devices. More particularly, the present invention relates to a pair of mobility devices adapted to be worn on the feet of a user and enable the user to walk on the ground at a faster speed without any skating movement or change in the user's gait pattern.

Commuters and other travelers often have to walk the final leg of their trip, regardless of whether they travelled by car, bus, train, or other means. Depending on the distance, the time needed to complete this final leg of the journey can comprise a significant amount of the total duration of the trip. While prior systems have utilized a control system in connection with wheeled, foot-worn mobility devices, these systems implemented motor controls that lacked precision or coordination with the user's actual movements. Therefore, it would be advantageous to develop a control system for a mobility device that provides improved control.

### BRIEF SUMMARY

According to embodiments of the present invention is a mobility device comprising a wheeled, motorized shoe for enabling pedestrians to walk faster without changing their natural gaits. In one embodiment, the motorized shoes add speed to the user's feet on the ground through rotational motion of wheels, which are driven by an electric motor connected to the wheels through a series of gears. The motorized shoes can brake by applying a braking torque from an electrical motor to the wheels through a gear train. In an alternative embodiment, the motorized shoes contain a separate braking mechanism. The motorized shoes can be adapted to the sole of normal shoes of a pedestrian; alternatively, the motorized shoes may be worn directly on the user's feet. A set of mechanical structures to allow natural rotation of the heel around the ball of a foot during normal walking.

The motorized shoes are controlled by an onboard control system comprising, in one embodiment, a main processor, a motor controller, inertia measurement units (IMU), a vision system, ultrasonic sensors, Global Position System Trackers (GPS), short ranged communication module, and Cellular/WiFi communication module.

The onboard control system may be operated in three different control configurations: Direct Control, Gait-Based Control and Cloud-Assisted Gait-Based Control. In Direct Control mode, the accelerations or speeds of each wheeled shoe is independently and directly controlled by a remote controller. In Gait-Based Control, a user can control the speeds of wheeled shoes based on their gait patterns. In this control mode, an algorithm calculates the pedestrian's stride length in real-time, maps the stride length to a pre-determined command speeds or accelerations and adjusts the command speeds based on the surrounding environment when the vision system is configured. In Cloud-Assisted Gait-Based Control mode, the control system authenticates the user's identification by uploading and crosschecking the user's gait features against a database in the cloud, in addition to performing the same operation as Gait-Based Control. In Cloud-Assisted Gait-Based Control mode, a fleet of the present inventions can operate in a shared mobility service network on demand.

### BRIEF SUMMARY OF THE SEVERAL VIEWS OF THE DRAWINGS

FIGS. 1A-1E depict various components of a motorized shoe, according to several embodiments.

FIGS. 2A-2B show fastening mechanisms, according to various embodiments, used to attached the motorized shoe to the foot of a user.

FIG. 3A is a functional diagram and the system architecture of the onboard control system.

FIG. 3B is an operation flowchart according to one control method.

FIG. 4A is a functional diagram and the system architecture of the onboard control system according to an alternative embodiment.

FIGS. 4B-4C are flow charts of various steps in the control method for the device depicted in FIG. 4A.

FIG. 5A is a functional diagram and the system architecture of the onboard control system according to an alternative embodiment.

FIG. 5B is a flow chart of various steps in the control method of the device depicted in FIG. 5A.

FIG. 6A is the network diagram of shared use environment.

FIG. 6B is the flowchart of operating a fleet of shared motorized shoes.

### DETAILED DESCRIPTION

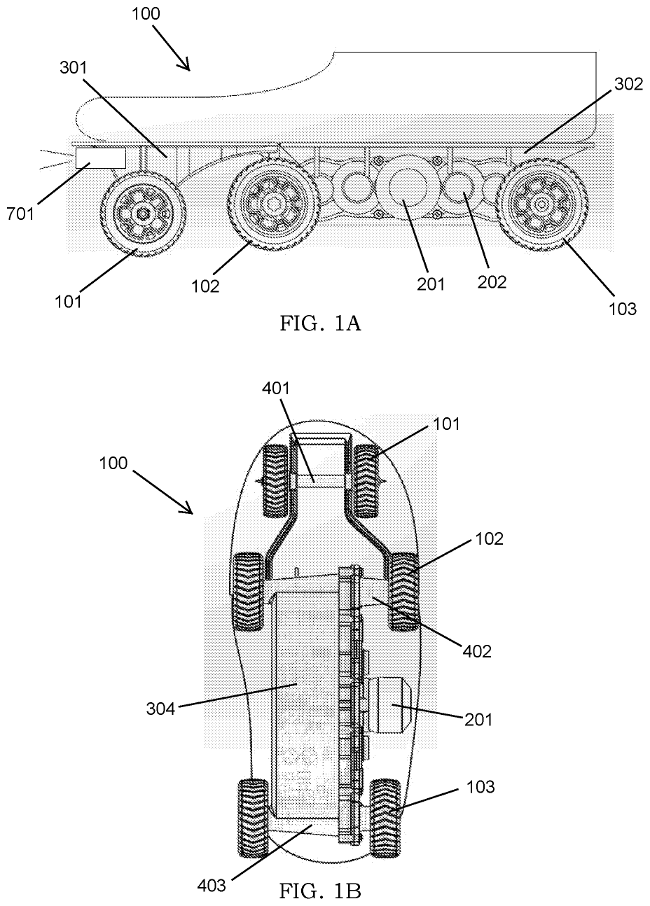

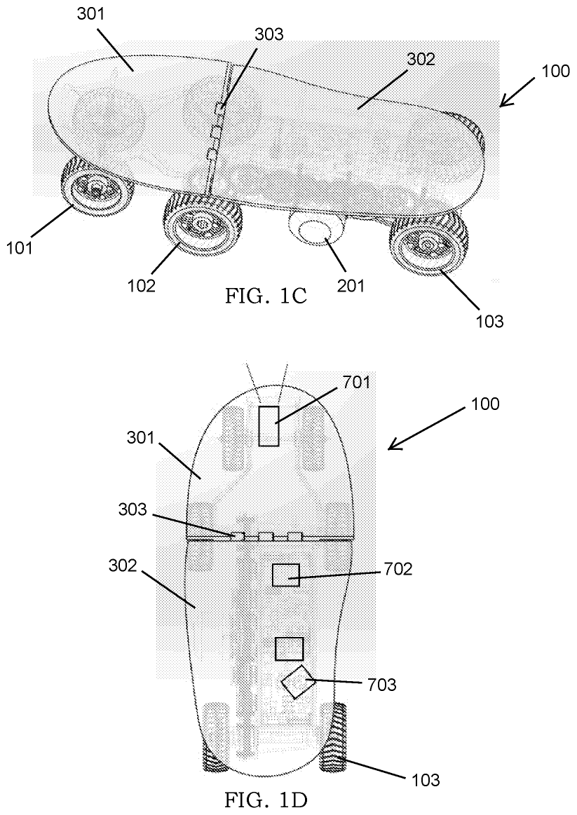

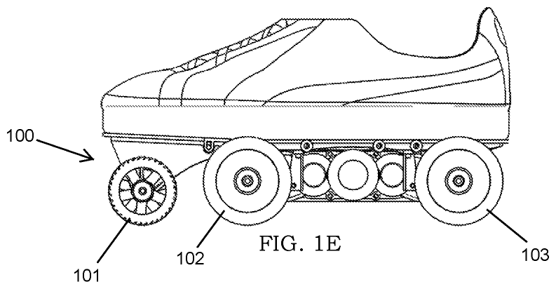

According to embodiments of the present invention, as shown in FIGS. 1A, 1B, 1C, is a motorized shoe 100 comprising multiple sets of wheels including a front set of wheels 101, a middle set of wheels 102, and a rear set of wheels 103. Both the middle set of wheels 102 and the rear set of wheels 103 are connected to an electric motor 201 through a geartrain 202. In one embodiment, the middle set of wheels 102 and rear set of wheels 103 have a larger diameter than the front set of wheels 101, where the top of the wheels extends beyond the top surface of a rear chassis 302, as shown in FIG. 1E. A rear chassis 302 provides a mounting point for the middle set of wheels 102 and the rear set of wheels 103. A front chassis 301 is connected to the rear chassis 302 by a pivoting member 303, such as a hinge, and provides a mounting point for the front set of wheels 101. The rear chassis 302 further integrates the geartrain 202, axle housings, and an electronics compartment 304. By incorporating these components into the rear chassis 302, the size and weight of the shoe 100 can be minimized. The electronics compartment 304 may include an onboard control system 700 and the battery pack. A user wears a pair of motorized shoes 101, one on each foot.

The entire foot of a user is supported by the front chassis 301 and rear chassis 302 and enables a user to walk faster by adding speed to their feet on ground, like walking on a moving walkway. In one embodiment, the motorized shoe 100 can brake effectively by applying a braking torque from the electrical motor 201 to certain wheels through the geartrain 202. Therefore, the stopping distance can be controlled by varying the amount of motor torques. In an alternative embodiment, a mechanical brake is provided and is connected to at least one of the first set of wheels 101, the middle set of wheels 102, or the rear set of wheels 103. The mechanical brake can be used by the control system 700, or the user can activate the mechanical brake in an emergency situation or as a kill-switch.

Referring to the axle configuration shown in FIG. 1B, the length of the middle axle 402 (supporting the middle set of wheels 102) is slightly larger than the girth of user's foot. The rear axle 403 (supporting the rear set of wheels 103) is slightly longer than the width of the user's heel, but is shorter than the middle wheel axle 402. The front axle 401 (supporting the front set of wheels 101) is the shortest to allow a foot twisting motion when the user turns.

As shown in FIG. 1C, to allow for natural walking motion of a foot, the front chassis 301 and the rear chassis 302 are inter-linked with a pivoting member 303, such as a lateral rod or hinge mechanism. In this configuration, the front chassis 301 and the rear chassis 302 can be rotated relative to each other around the ball of the user's foot. To further allow for a natural walking motion, the front set of wheels 101, although not connected to the gear train 202, are constrained to only rotate in the forward direction using anti-reverse bearings. As a result, when the pedestrian lifts his heel off the ground, the rear set of wheels 103 also lift off the ground and the rear chassis 302 is rotated around the pivoting member 303 relative to the front chassis 301. At the same time, the passive, or non-powered, front set of wheels 101 and the powered middle set of wheels 102 still provide traction and forward momentum by being in contact with the ground. As the user continues lifting his heel off the ground, the middle set of wheels 102 eventually lift off the ground. However, during this phase of the walking motion, the user's center of gravity has already been shifted to the other foot. Therefore, the passive front set of wheels 101 only need to ensure the motorized shoe 101 does not slip backward by constraining its rotational direction. The configuration in this embodiment allows for foot pivoting, provides sufficient resistance throughout the entire push off phase of the user's gait cycle, and simplifies the transmission by only connecting the middle set of wheel wheels 102 and the rear set of wheels 103 with the gear train 202.

Each shoe 101 may incorporate various components used by the control system 700. For example, as shown in FIG. 1D, a vision system 701 is installed in the front chassis 301 pointing in the forward direction, in the direction of travel of the user. In one embodiment, an ultrasonic sensor 703 is installed at the back end of the electronic compartment 304 inside the rear chassis 302, aligned at an angle from the forward direction. An inertia measurement unit 702 and a global position system tracker (GPS tracker) may also be installed in the electronic compartment 304 of the rear chassis 302.

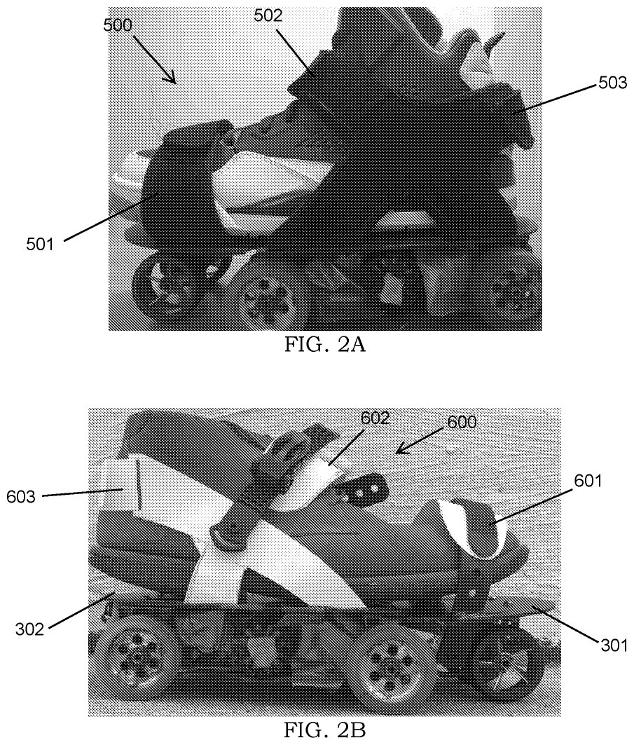

In one embodiment, the motorized shoes 101 are designed to fit over the shoe of the user. To secure the motorized shoe onto the shoe of the user, a hook-and-loop fastening system 500 is provided. The fastening system 500 shown in FIG. 2A comprises an adjustable front strap 501, which provides side-to-side and vertical constraint to the ball of a foot. The fastener 500 further comprises a main adjustable strap 502 providing vertical constraint to the ankle of a foot during the heel off phase of a gait cycle and an adjustable rear strap 503 providing longitudinal support to the heel of a foot, during the heel strike phase of a gait cycle.

FIG. 2B shows an alternative fastening system 600 utilizing a buckle strap construction to secure the user's shoe onto the top surface of the front chassis 301 and rear chassis 302. A person having skill in the art will appreciate that the design in this embodiment is similar to the construction of snowboard bindings and utilizes similar hardware. The fastening system 600 comprises a front strap 601 and an adjustable ratchet strap 602, which may include a padded element, and is positioned over the top of the foot. The ratchet strap 602 is attached to the rear chassis 302 with mechanical fasteners such as screws, rivets, or other methods. The fastening system further comprises an adjustable rear heel strap 603 and is made with soft/textile materials and is secured with hook and loop fastener.

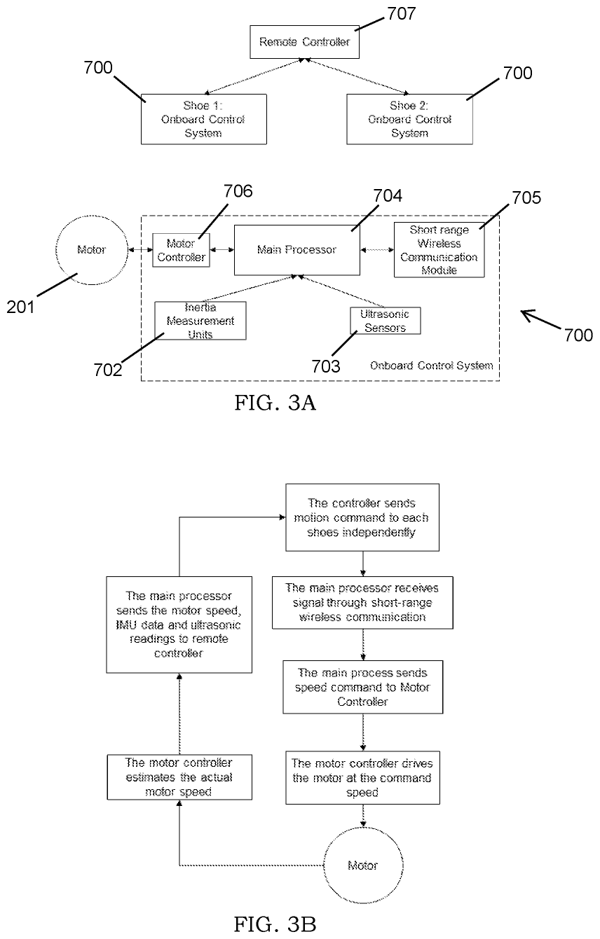

The shoes 100 are controlled by the onboard controller 700, with several different modes of control available. The hardware associated with an embodiment operating in a Direct Control mode is shown in FIG. 3A. As shown in the embodiment depicted in FIG. 3A, the controller 700 comprises a processor 704, a wireless communication module 705 (e.g. Bluetooth or Xbee), an ultrasonic sensor 703 (optional), an inertial measurement unit 702 (IMU) (optional), and a motor controller 706. Each shoe 101 will contain a controller 700. In addition, the shoes 101 may be connected by a remote controller 707, which can be used to activate braking in an emergency situation. The remote controller 707 is also used to send command speeds to both the left and right shoes 101. The remote controller 707 can be in the form of a hand-held controller, a computer, or a mobile phone.

FIG. 3B is a flowchart depicting the Direct Control mode of control. As shown in FIG. 3B, the remote controller 707 sends a motion command to each onboard control system 700 respectively. Once the main processor 704 receives the motion command, it converts the motion command into a speed command and signals the motor controller 706 to drive the motor 201 at the command speed.

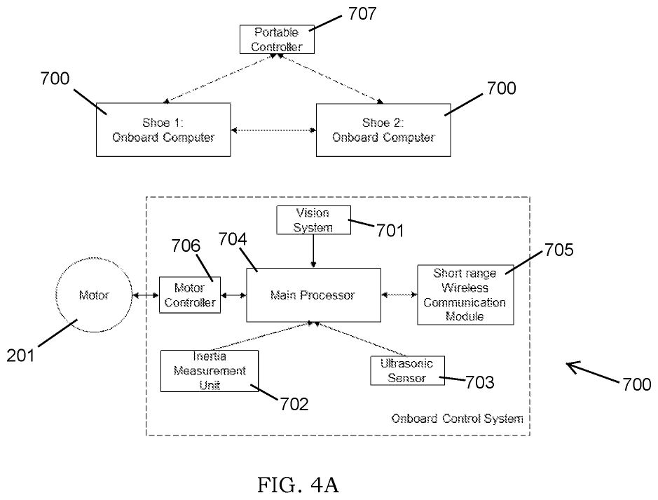

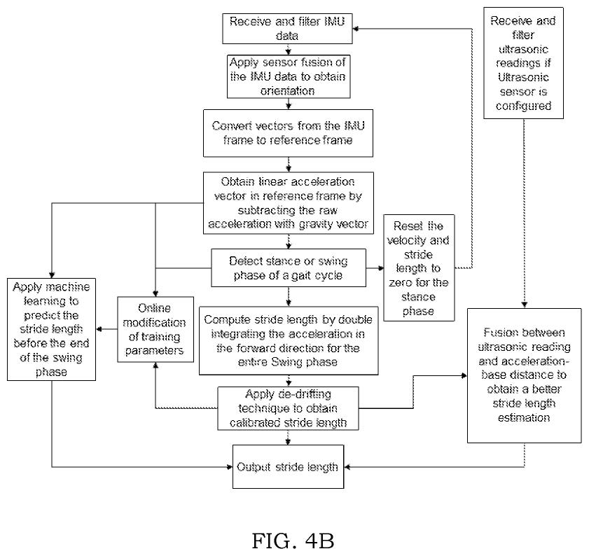

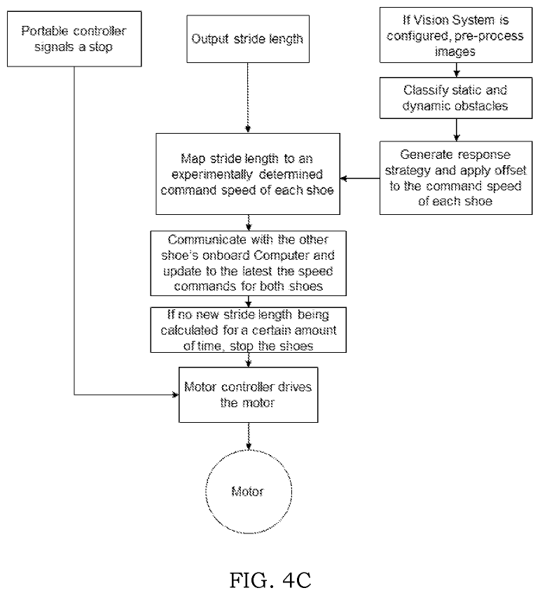

In an alternative embodiment, the shoes 100 are controlled in a Gait-Based Control mode. As shown in FIG. 4A, each onboard control system 700 utilized in the Gait-Based Control mode sets the command speed based on the estimation of the most recent stride length and communicates the command speed to the onboard control system 700 in the other shoe 100 of the pair in real time. A portable controller 707, which can be either worn or hand-held, can override the calculated command speed. The portable controller 707 can communicate with either one or both shoes 100 in real-time. Each onboard controller 700, in this embodiment, consists of a motor controller 706, a main processor 704, and a short range wireless communication module 705, inertia measurement units 702 with optional vision system 701, and ultrasonic sensors 703 installed.

As shown in FIGS. 4B and 4C, the Gait-Based Control mode comprises the steps of estimating stride length, mapping that stride length to speed commands, and communicating with the other motorized shoe 100 to ensure that both speed commands are updated with the latest stride length in real time. By way of further detail, after the main processor 704 in the controller 700 receives and filters IMU data, it applies a sensor fusion algorithm to the acceleration, gyroscopic, and magneto data to estimate the orientation of the motorized shoe 100. Once the orientation is estimated, the raw acceleration vector can be transformed from the IMU frame into the world frame. Next, the gravity vector can be subtracted from the acceleration vector in anteroposterior, lateral, and longitudinal directions to obtain linear acceleration. If the angular velocity (gyroscopic readings) around lateral axis is under a threshold and the sum of the squared accelerations in lateral and longitudinal directions is under a threshold, stance phase is detected, and the stride length is reset to zero. The swing phase is opposite of the stance phase. If in the swing phase of the gait cycle, the stride length is computed by double integrating the acceleration in the anteroposterior direction throughout the entire swing phase. As the velocities at both start and end instances of swing phase can be assumed zero, a linear de-drifting is applied to remove the drift during each stride length integration. When the optional ultrasonic sensors 703 are configured, the sensor reading is used to fuse with accelerated-based stride length to improve the accuracy of the stride length estimation. An output stride length is then generated.

FIG. 4C shows additional detail of the Gait-Based Control method. As shown in FIG. 4C, the output stride length is mapped to a command speed or acceleration for each shoe 100 through a pre-determined speed-to-stride length or acceleration-to-stride length relationship. If the last stride length was too short or no new stride length occurring for a certain amount of time, a stop command will be sent. The commands will then be used by the motor controller 707 to drive the motor 201. The purpose of these steps is to allow the user to control the speed of the shoes 100 with their own strides. In other words, when the user intends to accelerate, she can signal it by simply making larger strides. When the user intends to stop the present invention, she can stop walking. The speed to stride-length relationship can also be configured based on user preference.

Still referring to the Gait-Based Control as shown in FIG. 4B, a pre-trained machine learning algorithm takes in first few linear acceleration vectors and predicts the possible stride length before the end of the swing phase. During each swing phase, the more acceleration data the algorithm processes, the more accurate the predication is. Since every stride is always estimated, the algorithm parameters can be update online as each stride length calculation completes. Once the machine learning algorithm achieves comparable results as the double integration approach, the onboard control system 700 will start using the machine learning obtained stride length.

When the optional vision system 701 is configured, the algorithm classifies both static and dynamic obstacles into multiple response levels and applying offset to the command speeds, as shown in FIG. 4C. For example, if the algorithm determines the crack is too large for the shoes 100 to move across, it will gradually slow down the shoes. When the latest command speeds are computed on the shoes 100 in swing phase, they are executed by the motor controllers 706 either internally or via short-range communication.

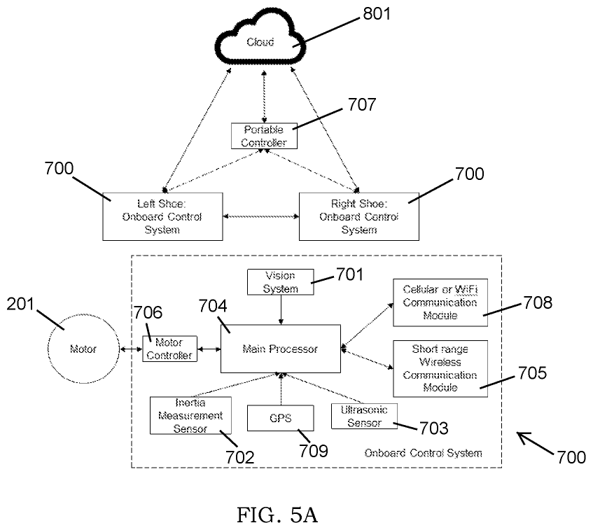

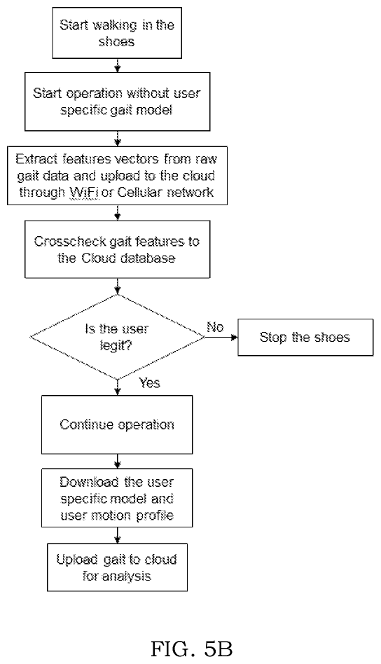

In yet another alternative embodiment, as shown in FIG. 5A, a Cloud Assisted Gait-Based Control mode is used to control the shoes 100. The onboard controller 700 in this mode comprises all the modules used in the Gait-Based Control mode in addition to a cellular or WiFi communication module 708 and GPS 709. The onboard control system 700 in this embodiment can communicate with the central could either directly or through the remote controller 707.

The Cloud Assisted Gait-based Control method comprises the steps of collecting gait data in real time, uploading processed gait features to the cloud, and using the gait information to verify user identification, in addition to the steps described in Gait-Based Control mode. For example, as shown in FIG. 5B, when a user start using the motorized shoes 100, it will commence collection of a user's gait features, processing the features, and crosschecking the features in the central cloud through a cellular or WiFi connection. If the user's identification is authenticated, the cloud will enable the shoes 100 to continue operation in the steps described in FIGS. 4B-4C. At the same time, the present invention will download the user's gait-model trained from using other units and the user's preferences. The user's gait features and trained gait-model are uploaded to the cloud at regular intervals.

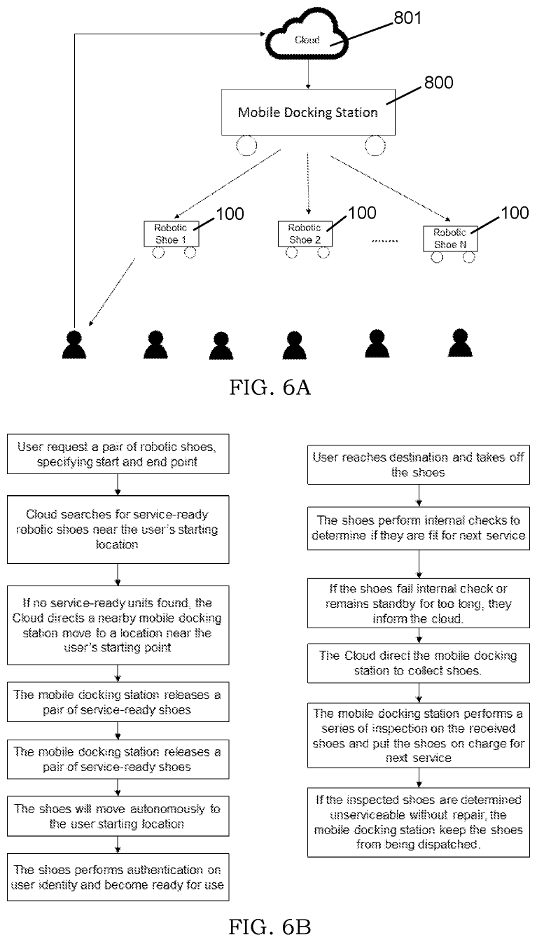

FIG. 6A shows the use of the shoes 100 on demand in a shared network. The network consists of users, multiple units of the motorized shoes 100, mobile docking stations 800, and the central cloud 801, which can be a central database, repository, server, or any combination of the foregoing. The mobile docking station 800 collects and dispatches the motorized shoes 100, charges their battery during docking, and conducts inspections on all received units.

FIG. 6B shows typical steps of using the motorized shoes 100 on demand in a shared manner. The process starts with a user requesting a pair of robotic shoes 100 and specifying the start and end of her upcoming trip. The cloud 801 then first tries to find a service-ready pair of robotic shoes 100 near the user's starting location. If no service-ready shoes 100 are found, the cloud 801 directs a nearby mobile docking station 800 to move to a location near the user's starting point. The mobile docking station 800 releases a pair of robotic shoes 100 some distance from the defined starting point and moves onto another target location immediately. The robotic shoes 100, equipped with an IMU 702, vision system 701, ultrasonic sensors 703, and GPS 709, complete the last leg of its dispatch journey to the user. The robotic shoes 100 will start authentication process as described in FIG. 5A as soon as user begins using the shoes 100. When the user reaches her destination and removes the shoes 100, the shoes 100 perform an internal check to determine if they are fit for next service without going to the docking station 800. If the shoes 100 fail the internal checks or remain in standby for too long, the shoes 100 inform the cloud 801, which will then direct a mobile docking station 801 to collect the shoes 100. The mobile docking station 801 collects the shoes 100, performs a series of inspections, and leaves the shoes 100 on charge for the next service.

1. A method of controlling a mobility device, the method comprising:
receiving, using a processor, inertial data from an inertial measurement unit, wherein the inertial data comprises a plurality of data vectors;
determining, based on the inertial data, an estimated orientation of the mobility device;
transforming, using the processor, at least one of the plurality of data vectors from a local frame to a world frame;
identifying, based on the transformed data vector, at least one phase of a gait cycle;
determining, based on the at least one phase, a stride length; and
generating, based on the determined stride length, an output stride length.

2. The method of claim 1, further comprising: predicting, based on the inertial data, an estimated stride length before the end of a gait cycle using a machine learning module.

3. The method of claim 1, further comprising:
obtaining, from an ultrasonic sensor, ultrasonic data; and
modifying, based on the inertial data and the ultrasonic data, the stride length.

4. The method of claim 1, further comprising obtaining, using a de-drifting technique, a calibrated stride length.

5. The method of claim 1, further comprising mapping the stride length to a command speed.

6. The method of claim 5, further comprising:
obtaining, from a vision system, one or more images;
identifying, based on the one or more images, one or more obstacles, wherein the one or more obstacles are selected from the group consisting of: a static obstacle and a dynamic obstacle;
generating, based on the one or more obstacles, a response strategy; and
applying, based on the response strategy, an offset to the command speed of the mobility device.

7. A mobility device adapted to be worn on the foot of a user comprising:
a rear chassis comprising:
a middle set of wheels; and
a rear set of wheels connected to an electric motor through a geartrain;
a front chassis comprising
a front set of wheels,
wherein the front chassis is connected to the rear chassis by a pivoting member; and
a control system configured to control the electric motor in response to an input from the user.

8. The mobility device of claim 7, wherein the control system comprises:
an inertial measurement unit and a processor.

9. The mobility device of claim 7, wherein the control system is housed in the rear chassis.

10. The mobility device of claim 7, wherein the middle set of wheels is connected by an axle, the axle having a width wider than a width of a user's foot.

11. The mobility device of claim 10, wherein the rear set of wheels is connected by an axle having a width smaller than the width of the axle connecting the middle set of wheels.

12. The mobility device of claim 11, wherein the front set of wheels is connected by an axle having a width smaller than the width of the axle connecting the rear set of wheels.

13. The mobility device of claim 7, further comprising one or more anti-reverse bearings associated with the front set of wheels.

14. The mobility device of claim 7, wherein the middle set of wheels and the rear set of wheels have a height greater than the height of the rear chassis.

15. The mobility device of claim 7, further comprising a mechanical brake connected to at least one of the front set of wheels, the middle set of wheels, or the rear set of wheels.

16. The mobility device of claim 15, wherein the mechanical brake is controlled by the control system.

17. The mobility device of claim 15, wherein the mechanical brake is controlled manually by a user.

18. The mobility device of claim 7, wherein the front chassis further comprises a rigid toe-cap.

19. The mobility device of claim 7, wherein the front chassis further comprises a semi-rigid toe-cap.
